(19) United States
(12) Patent Application Publication
(10) Pub. No.: US 2005/0227304 A1
(43) Pub. Date: Oct. 13, 2005

(54) Methods for the identification of inhibitors of histidinol dehydrogenase as antibiotics

(76) Inventors: Matthew M. Tanzer, Durham, NC (US); Jeffrey R. Shuster, Chapel Hill, NC (US); Lisbeth Hamer, Durham, NC (US); Kiichi Adachi, Osaka (JP); Todd M. DeZwaan, Apex, NC (US); Sze-Chung C. Lo, Hong Kong (CN); Maria Victoria Montenegro-Chamorro, Durham, NC (US); Blaise A. Darveaux, Hillsborough, NC (US); Sheryl A. Frank, Durham, NC (US); Ryan W. Heiniger, Holly Springs, NC (US); Sanjoy K. Mahanty, Chapel Hill, NC (US); Huaqin Pan, Apex, NC (US); Amy S. Covington, Raleigh, NC (US); Rex Tarpey, Apex, NC (US)

Correspondence Address: ERIC J. KRON;ICORIA, INC., 108 T.W. ALEXANDER DRIVE, BUILDING 1A, POST OFFICE BOX 14528, RESEARCH TRIANGLE PARK, NC, 27709, US

(21) Appl. No.: 11/076,770

(22) Filed: Mar. 10, 2005

Related U.S. Application Data
(60) Provisional application No. 60/552,514, filed on Mar. 12, 2004.

(51) Int. Cl. C12Q 1/68; G01N 33/53; G01N 33/569; C12Q 1/18

(52) U.S. Cl. 435/7.31; 435/32

(57) ABSTRACT

The present inventors have discovered that a histidinol dehydrogenase (HIS4) is essential for normal fungal pathogenicity, Specifically, the inhibition of HIS4 gene expression in Magnaportha grisea severely reduces the pathogenicity of the fungus. Thus, HIS4 is useful as a target for the identification of antibiotics, preferably fungicides. Accordingly, the present invention provides methods for the identification of compounds that inhibit HIS4 expression or activity. The methods of the invention are useful for the identification of antibiotics, preferably fungicides.

### RELATED APPLICATIONS

This application claims the benefit of U.S. Provisional Application No. 60/552,514, filed on Mar. 12, 2004, which is incorporated in its entirety by reference.

### FIELD OF THE INVENTION

The invention relates generally to methods for the identification of antibiotics.

### BACKGROUND OF THE INVENTION

Filamentous fungi are causal agents responsible for many serious pathogenic infections of plants and animals. Since fungi are eukaryotes, and thus more similar to their host organisms than, for example bacteria, the treatment of infections by fungi poses special risks and challenges not encountered with other types of infections. One such fungus is Magnaporthe grisea, the fungus that causes rice blast disease, a significant threat to food supplies worldwide. Other examples of plant pathogens of economic importance include the pathogens in the genera Agaricus, Alternaria, Anisogramma, Anthracoidea, Antrodia, Apiognomonia, Apiosporina, Armillaria, Ascochyta, Aspergillus, Bipolaris, Bjerkandera, Botryosphaeria, Botrytis, Ceratobasidium, Ceratocystis, Cercospora, Cercosporidium, Cerotelium, Cerrena, Chondrostereum, Chryphonectria, Chrysomyxa, Cladosporium, Claviceps, Cochliobolus, Coleosporium, Colletotrichium, Colletotrichum, Corticium, Corynespora, Cronartium, Cryphonectria, Cryptosphaeria, Cyathus, Cymadothea, Cytospora, Daedaleopsis, Diaporthe, Didymella, Diplocarpon, Diplodia, Discohainesia, Discula, Dothistroma, Drechslera, Echinodontium, Elsinoe, Endocronartium, Endothia, Entyloma, Epichloe, Erysiphe, Exobasidium, Exserohilum, Fomes, Fomitopsis, Fusarium, Gaeumannomyces, Ganoderma, Gibberella, Gloeocercospora, Gloeophyllum, Gloeoporus, Glomerella, Gnomoniella, Guignardia, Gymnosporangium, Helm inthosporium, Herpotrichia, Heterobasidion, Hirsch ioporus, Hypodermella, Inonotus, Irpex, Kabatiella, Kabatina, Laetiporus, Laetisaria, Lasiodiplodia, Laxitextum, Leptographium, Leptosphaeria, Leptosphaerulina, Leucytospora, Linospora, Lophodermella, Lophodermium, Macrophomina, Magnaporthe, Marssonina, Melampsora, Melampsorella, Meria, Microdochium, Microsphaera, Monilinia, Monochaetia, Morchella, Mycosphaerella, Myrothecium, Nectria, Nigrospora, Ophiosphaerella, Ophiostoma, Penicillium, Perenniporia, Peridermium, Pestalotia, Phaeocryptopus, Phaeolus, Phakopsora, Phellinus, Phialophora, Phoma, Phomopsis, Phragmidium, Phyllachora, Phyllactinia, Phyllosticta, Phymatotrichopsis, Pleospora, Podosphaera, Pseudopeziza, Pseudoseptoria, Puccinia, Pucciniastrum, Pyricularia, Rhabdocline, Rhizoctonid, Rhizopus, Rhizosphaera, Rhynchosporium, Rhytisma, Schizophyllum, Schizopora, Scirrhia, Sclerotinia, Sclerotium, Scytinostroma, Septoria, Setosphaera, Sirococcus, Spaerotheca, Sphaeropsis, Sphaerotheca, Sporisorium, Stagonospora, Stemphylium, Stenocarpella, Stereum, Taphrina, Thielaviopsis, Tilletia, Trametes, Tranzschelia, Trichoderma, Tubakia, Typhula, Uncinula, Urocystis, Uromyces, Ustilago, Valsa, Venturia, Verticillium, Xylaria, and others. Related organisms are classified in the oomycetes classification and include the genera Albugo, Aphanomyces, Bremia, Peronospora, Phytophthora, Plasmodiophora, Plasmopara, Pseudoperonospora, Pythium, Sclerophthora, and others. Oomycetes are also significant plant pathogens and are sometimes classified along with the true fungi.

Human diseases that are caused by filamentous fungi include life-threatening lung and disseminated diseases, often a result of infections by Aspergillus fumigatus. Other fungal diseases in animals are caused by fungi in the genera Fusarium, Blastomyces, Microsporum, Trichophyton, Epidermophyton, Candida, Histoplamsa, Pneumocystis, Cryptococcus, other Aspergilli, and others. Control of fungal diseases in plants and animals is usually mediated by chemicals that inhibit growth, proliferation, and/or pathogenicity of fungal organisms. To date, there are less than twenty known modes-of-action for plant protection fungicides and human antifungal compounds.

A pathogenic organism has been defined as an organism that causes, or is capable of causing disease. Pathogenic organisms propagate on or in tissues and may obtain nutrients and other essential materials from their hosts. A substantial amount of work concerning filamentous fungal pathogens has been performed with the human pathogen, Aspergillus fumigatus. Shibuya et al, 27 Microb. Pathog. 123 (1999) (PubMed Identifier (PMID): 10455003) have shown that the deletion of either of two suspected pathogenicity related genes encoding an alkaline protease or a hydrophobin (rodlet), respectively, did not reduce mortality of mice infected with these mutant strains. Smith et al, 62 Infect. Immun. 5247 (1994) (PMID: 7960101) showed similar results with alkaline protease and the ribotoxin restrictocin; Aspergillus fumigatus strains mutated for either of these genes were fully pathogenic to mice. Reichard et al., 35 J. Med. Vet. Mycol. 189 (1997) (PMID: 9229335) showed that deletion of the suspected pathogenicity gene encoding aspergillopepsin (PEP) in Aspergillus fumigatus had no effect on mortality in a guinea pig model system, whereas Aufauvre-Brown et al., 21 Fungal. Genet. Biol. 141 (1997) (PMID: 9073488) showed no effects of a chitin synthase mutation on pathogenicity.

However, not all experiments produced negative results. Ergosterol is an important membrane component found in fungal organisms. Pathogenic fungi lacking key enzymes in the ergosterol biochemical pathway might be expected to be non-pathogenic since neither the plant nor animal hosts contain this particular sterol. Many antifungal compounds that affect the ergosterol biochemical pathway have been previously described. (U.S. Pat. Nos. 4,920,109; 4,920,111; 4,920,112; 4,920,113; and 4,921,844; Hewitt, H. G. Fungicides in Crop Protection Cambridge, University Press (1998)). D'Enfert et al., 64 Infect. Immun. 4401 (1996) (PMID: 8926121)) showed that an Aspergillus fumigatus strain mutated in an orotidine 5′-phosphate decarboxylase gene was entirely non-pathogenic in mice, and Brown et al. (Brown et al., 36 Mol. Microbiol. 1371 (2000) (PMID: 10931287)) observed a non-pathogenic result when genes involved in the synthesis of para-aminobenzoic acid were mutated. Some specific target genes have been described as having utility for the screening of inhibitors of plant pathogenic fungi. U.S. Pat. No. 6,074,830 to Bacot et al., describe the use of 3,4-dihydroxy-2-butanone 4-phosphate synthase, and U.S. Pat. No. 5,976,848 to Davis et al. describes the use of dihydroorotate dehydrogenase for potential screening purposes.

There are also a number of papers that report less clear results, showing neither full pathogenicity nor non-pathogenicity of mutants. For example, Hensel et al. (Hensel, M. et al., 258 Mol. Gen. Genet. 553 (1998) (PMID: 9669338)) showed only moderate effects of the deletion of the areA transcriptional activator on the pathogenicity of Aspergillus fumigatus. Therefore, it is not currently possible to determine which specific growth materials may be readily obtained by a pathogen from its host, and which materials may not.

The present invention discloses histidinol dehydrogenase as a target for the identification of antifungal, biocide, and biostatic materials.

### SUMMARY OF THE INVENTION

The present inventors have discovered that in vivo disruption of the gene encoding a histidinol dehydrogenase in Magnaporthe grisea eliminates the pathogenicity of the fungus. Thus, the present inventors have discovered that the histidinol dehydrogenase is useful as a target for the identification of antibiotics, preferably fungicides. Accordingly, the present invention provides methods for the identification of compounds that inhibit the histidinol dehydrogenase expression or activity. Methods of the invention are useful for the identification of antibiotics, preferably fungicides.

### BRIEF DESCRIPTION OF THE FIGURES

FIG. 1. Digital image showing the effect of HIS4 gene disruptions on Magnaporthe grisea pathogenicity using whole plant infection assays. Rice variety CO₃₉ was inoculated with wild-type strain Guy11 and cpgmra0037002c06 transposon insertion strains K1-38 and K1-48. Leaf segments were imaged at five days post-inoculation.

### DETAILED DESCRIPTION OF THE INVENTION

Unless otherwise indicated, the following terms are intended to have the following meanings in interpreting the present invention.

The term “antibiotic” refers to any substance or compound that when contacted with a living cell, organism, virus, or other entity capable of replication, results in a reduction of growth, viability, or pathogenicity of that entity.

The term “antipathogenic,” as used herein, refers to a mutant form of a gene that inactivates a pathogenic activity of an organism on its host organism or substantially reduces the level of pathogenic activity, wherein “substantially” means a reduction at least as great as the standard deviation for a measurement, preferably a reduction to 50% activity, more preferably a reduction of at least one magnitude, i.e. to 10% activity. The pathogenic activity affected may be an aspect of pathogenic activity governed by the normal form of the gene, or the pathway the normal form of the gene functions on, or the pathogenic activity of the organism in general. “Antipathogenic” may also refer to a cell, cells, tissue, or organism that contains the mutant form of a gene; a phenotype associated with the mutant form of a gene, and/or associated with a cell, cells, tissue, or organism that contain the mutant form of a gene.

The term “binding” refers to a non-covalent or a covalent interaction, preferably non-covalent, that holds two molecules together. For example, two such molecules could be an enzyme and an inhibitor of that enzyme. Non-covalent interactions include hydrogen bonding, ionic interactions among charged groups, van der Waals interactions, and hydrophobic interactions among nonpolar groups. One or more of these interactions can mediate the binding of two molecules to each other.

The term “biochemical pathway” or “pathway” refers to a connected series of biochemical reactions normally occurring in a cell. Typically, the steps in such a biochemical pathway act in a coordinated fashion to produce a specific product or products or to produce some other particular biochemical action. Such a biochemical pathway requires the expression product of a gene if the absence of that expression product either directly or indirectly prevents the completion of one or more steps in that pathway, thereby preventing or significantly reducing the production of one or more normal products or effects of that pathway. Thus, an agent specifically inhibits such a biochemical pathway requiring the expression product of a particular gene if the presence of the agent stops or substantially reduces the completion of the series of steps in that pathway. Such an agent may, but does not necessarily, act directly on the expression product of that particular gene.

As used herein, the term “conditional lethal” refers to a mutation permitting growth and/or survival only under special growth or environmental conditions.

As used herein, the term “cosmid” refers to a hybrid vector used in gene cloning that includes a cos site (from the lambda bacteriophage). In some cases, the cosmids of the invention comprise drug resistance marker genes and other plasmid genes. Cosmids are especially suitable for cloning large genes or multigene fragments.

“Fungi” (singular: fungus) refers to whole fungi, fungal organs and tissues (e.g., asci, hyphae, pseudohyphae, rhizoid, sclerotia, sterigmata, spores, sporodochia, sporangia, synnemata, conidia, ascostroma, cleistothecia, mycelia, perithecia, basidia and the like), spores, fungal cells and the progeny thereof. Fungi are a group of organisms (about 50,000 known species), including, but not limited to, mushrooms, mildews, moulds, yeasts, etc., comprising the kingdom Fungi. Fungi exist as single cells or a multicellular body called a mycelium, which consists of filaments known as hyphae. Most fungal cells are multinucleate and have cell walls composed chiefly of chitin. Fungi exist primarily in damp situations on land, and lacking the ability to manufacture their own food by photosynthesis due to the absence of chlorophyll, are either parasites on other organisms or saprotrophs feeding on dead organic matter. Principal criteria used in classification are the nature of the spores produced and the presence or absence of cross walls within the hyphae. Fungi are distributed worldwide in terrestrial, freshwater, and marine habitats. Some fungi live in the soil. Many pathogenic fungi cause disease in animals and man or in plants, while some saprotrophs are destructive to timber, textiles, and other materials. Some fungi form associations with other organisms, most notably with algae to form lichens.

As used herein, the term “fungicide,” “antifungal,” or “antimycotic” refers to an antibiotic substance or compound that kills or suppresses the growth, viability, or pathogenicity of at least one fungus, fungal cell, fungal tissue or spore.

In the context of this disclosure, “gene” should be understood to refer to a unit of heredity. Each gene is composed of a linear chain of deoxyribonucleotides that can be referred to by the sequence of nucleotides forming the chain. Thus, “sequence” is used to indicate both the ordered listing of the nucleotides that form the chain, and the chain having that sequence of nucleotides. “Sequence” is used in the similar way in referring to RNA chains, linear chains made of ribonucleotides. The gene may include regulatory and control sequences, sequences that can be transcribed into an RNA molecule, and may contain sequences with unknown function. The majority of the RNA transcription products are messenger RNAs (mRNAs), which include sequences that are translated into polypeptides and may include sequences that are not translated. It should be recognized that small differences in nucleotide sequence for the same gene can exist between different fungal strains, or even within a particular fungal strain, without altering the conservation of the gene.

As used in this disclosure, the terms “growth” or “cell growth” of an organism refer to an increase in mass, density, or number of cells of the organism. Common methods for the measurement of growth include the determination of the optical density of a cell suspension, the counting of the number of cells in a fixed volume, the counting of the number of cells by measurement of cell division, the measurement of cellular mass or cellular volume, and the like.

As used in this disclosure, the term “growth conditional phenotype” indicates that a fungal strain having such a phenotype exhibits a significantly greater difference in growth rates in response to a change in one or more of the culture parameters than an otherwise similar strain not having a growth conditional phenotype. Typically, a growth conditional phenotype is described with respect to a single growth culture parameter, such as temperature. Thus, a temperature (or heat-sensitive) mutant (i.e., a fungal strain having a heat-sensitive phenotype) exhibits significantly different growth, and preferably no growth, under non-permissive temperature conditions as compared to growth under permissive conditions. In addition, such mutants preferably also show intermediate growth rates at intermediate, or semi-permissive, temperatures. Similar responses also result from the appropriate growth changes for other types of growth conditional phenotypes.

As used herein, the terms “heterologous HIS4” and “heterologous histidinol dehydrogenase” mean either a nucleic acid encoding a polypeptide or a polypeptide, wherein the polypeptide has at least 40%, 45%, 50%, 55%, 60%, 65%, 70%, 75%, 80%, 85%, 90%, 91%, 92%, 93%, 94%, 95%, 96%, 97%, 98%, or 99% sequence conservation or each integer unit of sequence conservation from 40-100% in ascending order to M. grisea histidinol dehydrogenase protein (SEQ ID NO:2) and at least 10%, 25%, 50%, 75%, 80%, 90%, 95%, or 99% activity or each integer unit of activity from 10-100% in ascending order of the activity of M. grisea histidinol dehydrogenase protein (SEQ ID NO:2). Examples of heterologous histidinol dehydrogenases include, but are not limited to, HIS4 from Neurospora crassa (swissprot Accession No.: P07685), Candida albicans (swissprot Accession No.: 074712) and Pichia pastoris (swissprot Accession No.: P45353).

As used herein, the term “His-Tag” refers to an encoded polypeptide consisting of multiple consecutive histidine amino acids.

As used herein, the terms “histidinol dehydrogenase,” “HIS4 polypeptide,”“HIS4,” and “HIS4 gene product” are used interchangeably and refer to a polypeptide that catalyzes the reversible inter-conversion of L-histidinol, 2 NAD⁺ and H₂O to L-histidine and 2 NADH. Although the name of the protein and/or the name of the gene that encodes the protein may differ between species, the terms “histidinol dehydrogenase” and “HIS4 gene product” are intended to encompass any polypeptide that catalyzes the foregoing reaction.

As used herein, the terms “hph,” “hygromycin B phosphotransferase,” and “hygromycin resistance gene” refer to a hygromycin phosphotransferase gene or gene product.

As used herein, the term “imperfect state” refers to a classification of a fungal organism having no demonstrable sexual life stage.

The term “inhibitor,” as used herein, refers to a chemical substance that interferes with HIS4 function, such as interfering with and/or inactivating or substantially reducing the activity of histidinol dehydrogenase, wherein “substantially” means a reduction at least as great as the standard deviation for a measurement, preferably a reduction to 50% activity, more preferably a reduction of at least one magnitude, i.e. to 10% activity. The inhibitor may function by interacting directly with the enzyme, a cofactor of the enzyme, the substrate of the enzyme, or any combination thereof.

A polynucleotide may be “introduced” into a fungal cell by any means known to those of skill in the art, including transfection, transformation or transduction, transposable element, electroporation, particle bombardment, infection, and the like. The introduced polynucleotide may be maintained in the cell stably if it is incorporated into a non-chromosomal autonomous replicon or integrated into the fungal chromosome. Alternatively, the introduced polynucleotide may be present on an extra-chromosomal non-replicating vector and be transiently expressed or transiently active.

As used herein, the term “knockout” or “gene disruption” refers to the creation of organisms carrying a null mutation (a mutation in which there is no active gene product), a partial null mutation or mutations, or an alteration or alterations in gene regulation by interrupting a DNA sequence through insertion of a foreign piece of DNA. Usually the foreign DNA encodes a selectable marker.

As used herein, the term “mutant form” of a gene refers to a gene that has been altered, either naturally or artificially, by changing the base sequence of the gene. The change in the base sequence may be of several different types, including changes of one or more bases for different bases, deletions, and/or insertions, such as by a transposon. In contrast, a normal form of a gene (wild-type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “Ni-NTA” refers to nickel sepharose.

As used herein, a “normal” form of a gene (wild-type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “pathogenicity” refers to a capability of causing disease and/or degree of capacity to cause disease. The term is applied to parasitic micro-organisms in relation to their hosts. As used herein, “pathogenicity,” “pathogenic,” and the like, encompass the general capability of causing disease as well as various mechanisms and structural and/or functional deviations from normal used in the art to describe the causative factors and/or mechanisms, presence, pathology, and/or progress of disease, such as virulence, host recognition, cell wall degradation, toxin production, infection hyphae, penetration peg production, appressorium production, lesion formation, sporulation, and the like.

The “percent (%) sequence conservation” between two polynucleotide or two polypeptide sequences is determined according to either the BLAST program (Basic Local Alignment Search Tool, (Altschul, S. F. et al., 215 J. Mol. Biol. 403 (1990) (PMID: 2231712)) or using Smith Waterman Alignment (T. F. Smith & M. S. Waterman 147 J. Mol. Biol. 195. (1981) (PMID: 7265238)). It is understood that for the purposes of determining sequence conservation when comparing a DNA sequence to an RNA sequence, a thymine nucleotide is equivalent to a uracil nucleotide.

By “polypeptide” is meant a chain of at least two amino acids joined by peptide bonds. The chain may be linear, branched, circular or combinations thereof. The polypeptides may contain amino acid analogs and other modifications, including, but not limited to glycosylated or phosphorylated residues.

As used herein, the term “proliferation” is synonymous to the term “growth.”

As used herein, “semi-permissive conditions” are conditions in which the relevant culture parameter for a particular growth conditional phenotype is intermediate between permissive conditions and non-permissive conditions. Consequently, in semi-permissive conditions an organism having a growth conditional phenotype will exhibit growth rates intermediate between those shown in permissive conditions and non-permissive conditions. In general, such intermediate growth rate may be due to a mutant cellular component that is partially functional under semi-permissive conditions, essentially fully functional under permissive conditions, and is non-functional or has very low function under non-permissive conditions, where the level of function of that component is related to the growth rate of the organism. An intermediate growth rate may also be a result of a nutrient substance or substances that are present in amounts not sufficient for optimal growth rates to be achieved.

“Sensitivity phenotype” refers to a phenotype that exhibits either hypersensitivity or hyposensitivity.

The term “specific binding” refers to an interaction between a histidinol dehydrogenase (HIS4) and a molecule or compound, wherein the interaction is dependent upon the primary amino acid sequence and/or the tertiary conformation of the histidinol dehydrogenase.

“Transform,” as used herein, refers to the introduction of a polynucleotide (single or double stranded DNA, RNA, or a combination thereof) into a living cell by any means. Transformation may be accomplished by a variety of methods, including, but not limited to, electroporation, polyethylene glycol mediated uptake, particle bombardment, agrotransformation, and the like. The transformation process may result in transient or stable expression of the transformed polynucleotide. By “stably transformed” is meant that the sequence of interest is integrated into a replicon in the cell, such as a chromosome or episome. Transformed cells encompass not only the end product of a transformation process, but also the progeny thereof, which retain the polynucleotide of interest.

For the purposes of the invention, “transgenic” refers to any cell, spore, tissue or part that contains all or part of at least one recombinant polynucleotide. In many cases, all or part of the recombinant polynucleotide is stably integrated into a chromosome or stable extra-chromosomal element, so that it is passed on to successive generations.

As used herein, the term “Tween 20” means sorbitan mono-9-octadecenoate poly(oxy-1,1-ethanediyl).

As used in this disclosure, the term “viability” of an organism refers to the ability of an organism to demonstrate growth under conditions appropriate for the organism, or to demonstrate an active cellular function. Some examples of active cellular functions include respiration as measured by gas evolution, secretion of proteins and/or other compounds, dye exclusion, mobility, dye oxidation, dye reduction, pigment production, changes in medium acidity, and the like.

The present inventors have discovered that disruption of the HIS4 gene in Magnaporthe grisea drastically reduces pathogenicity of the fungus. Thus, the inventors demonstrate that the HIS4 gene product is a target for antibiotics, preferably fungicides. Accordingly, the invention provides methods for identifying compounds that inhibit HIS4 gene expression or biological activity of its gene product(s). Such methods include ligand-binding assays, assays for enzyme activity, cell-based assays, and assays for HIS4 gene expression. The compounds identified by the methods of the invention are useful as antibiotics.

Thus, in one embodiment, the invention provides a method for identifying a test compound as a candidate for an antibiotic, comprising contacting a HIS4 polypeptide with a test compound and detecting the presence or absence of binding between the test compound and the HIS4 polypeptide, wherein binding indicates that the test compound is a candidate for an antibiotic. HIS4 polypeptides of the invention have the amino acid sequence of naturally occurring HIS4 polypeptides found in a fungus, animal, plant, or microorganism, or have an amino acid sequence derived from a naturally occurring sequence. Preferably the HIS4 is a fungal HIS4. A cDNA encoding M. grisea HIS4 protein is set forth in SEQ ID NO: 1 and a M. grisea HIS4 polypeptide is set forth in SEQ ID NO:2. The genomic DNA encoding the M. grisea HIS4 protein is set forth in SEQ ID NO:3. In one embodiment, the HIS4 is a Magnaporthe HIS4. Magnaporthe species include, but are not limited to, Magnaporthe rhizophila, Magnaporthe salvinii, Magnaporthe grisea, Magnaporthe oryzae and Magnaporthe poae and the imperfect states of Magnaporthe in the genus Pyricularia. Preferably, the Magnaporthe HIS4 is from Magnaporthe grisea.

In one embodiment, the invention provides a polypeptide consisting essentially of SEQ ID NO:2. For the purposes of the present invention, a polypeptide consisting essentially of SEQ ID NO:2 has at least 90% sequence identity with M. grisea HIS4 (SEQ ID NO:2) and at least 10% of the activity of SEQ ID NO:2. A polypeptide consisting essentially of SEQ ID NO:2 has at least 90%, 91%, 92%, 93%, 94%, 95%, 96%, 97%, 98%, or 99% sequence identity with SEQ ID NO:2 and at least 25%, 50%, 75%, or 90% of the activity of M. grisea HIS4. Examples of polypeptides consisting essentially of SEQ ID NO:2 include, but are not limited to, polypeptides having the amino acid sequence of SEQ ID NO:2 with the exception that one or more of the amino acids are substituted with structurally similar amino acids providing a conservative amino acid substitution. Conservative amino acid substitutions are well known to those of skill in the art. Examples of polypeptides consisting essentially of SEQ ID NO:2 include polypeptides having 1, 2, or 3 conservative amino acid substitutions relative to SEQ ID NO:2. Other examples of polypeptides consisting essentially of SEQ ID NO:2 include polypeptides having the sequence of SEQ ID NO:2, but with truncations at either or both the 3′ and the 5′ end. For example, polypeptides consisting essentially of SEQ ID NO:2 include polypeptides having 1, 2, or 3 amino acids residues removed from either or both 3′ and 5′ ends relative to SEQ ID NO:2.

In various embodiments, the HIS4 gene product can be from Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus, (Monilinia fructigena), Apple-rotting Fungus (Penicillium expansum) Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), and the like.

Fragments of a HIS4 polypeptide are useful in the methods of the invention. In one embodiment, the HIS4 fragments include an intact or nearly intact epitope that occurs on biologically active wild-type HIS4. For example, the fragments comprise at least 10 consecutive amino acids of HIS4 set forth in SEQ ID NO:2. The fragments comprise at least 15, 20, 25, 30, 35, 40, 50, 60, 70, 80, 90, 100, 125, 150, 175, 200, 225, 250, 300, 325, 350, 375, 400, 425, 450, 475, 500, 525, 550, 575, 600, 625, 650, 675, 700, 725, 750, 775, 800, 825 or at least 850 consecutive amino acids residues of HIS4 set forth in SEQ ID NO:2. Fragments of heterologous HIS4's are also useful in the methods of the invention. For example, polypeptides having at least 50%, 60%, 70%, 80%, 90%, 95%, 96%, 97%, 98% or 99% sequence conservation with at least 50 consecutive amino acid residues of SEQ ID NO:2 are useful in the methods of the invention. In one embodiment, the fragment is from a Magnaporthe HIS4. In an alternate embodiment, the fragment contains an amino acid sequence conserved between fungal HIS4's. Particularly useful fragments of the invention are fragments of HIS4 proteins that include an intact or nearly intact epitope present at a non-membrane spanning region of a biologically active HIS4 protein. For example, such a fragment comprises at least 10 consecutive amino acids occurring at a non-membrane spanning region of the HIS4 protein set forth in SEQ ID NO:2. Procedures for identifying non-membrane spanning regions of proteins, such as the HIS4 proteins of the invention, based on analysis of the polypeptide sequence for conserved signal-processing and membrane spanning sequences are known to those of ordinary skill in the art.

Polypeptides having at least 40% sequence conservation with M. grisea HIS4 (SEQ ID NO:2) protein are also useful in the methods of the invention. In one embodiment, the sequence conservation is at least 40%, 45%, 50%, 55%, 60%, 65%, 70%, 75%, 80%, 85%, 90%, 91%, 92%, 93%, 94%, 95%, 96%, 97%, 98% or 99%, or any integer from 40-100% sequence conservation in ascending order with M. grisea HIS4 (SEQ ID NO:2) protein. In addition, it is preferred that polypeptides of the invention have at least 10% of the activity of M. grisea HIS4 (SEQ ID NO:2) protein. HIS4 polypeptides of the invention have at least 10%, 15%, 20%, 25%, 30%, 35%, 40%, 45%, 50%, 55%, 60%, 65%, 70%, 75%, 80%, 85% or at least 90% of the activity of M grisea HIS4 (SEQ ID NO:2) protein.

Thus, in another embodiment, the invention provides a method for identifying a test compound as a candidate for a fungicide, comprising: contacting a test compound with at least one polypeptide selected from the group consisting of: a polypeptide consisting essentially of SEQ ID NO:2, a polypeptide having at least ten consecutive amino acids of SEQ ID NO:2, a polypeptide having at least 40% sequence conservation with SEQ ID NO:2 and at least 10% of the activity of SEQ ID NO:2, and a polypeptide consisting of at least 50 amino acids having at least 50% sequence conservation with SEQ ID NO:2 and at least 10% of the activity of SEQ ID NO:2, and detecting the presence and/or absence of binding between the test compound and the polypeptide, wherein binding indicates that the test compound is a candidate for an antibiotic.

Any technique for detecting the binding of a ligand to its target may be used in the methods of the invention. For example, the ligand and target are combined in a buffer. Many methods for detecting the binding of a ligand to its target are known in the art, and include, but are not limited to, the detection of an immobilized ligand-target complex or the detection of a change in the properties of a target when it is bound to a ligand. In a preferred embodiment, bound HIS4 is detected using a labeled binding partner, such as a labeled antibody. In an alternate preferred embodiment, HIS4 is labeled prior to contacting the immobilized candidate ligands. Preferred labels include fluorescent or radioactive moieties. Preferred detection methods include fluorescence correlation spectroscopy (FCS), FCS-related confocal nanofluorimetric methods, and liquid scintillation counting.

In another embodiment of the invention compounds are identified as candidates for antibiotics by their ability to inhibit HIS4 enzymatic activity. The compounds are tested using either in vitro or cell based assays. Alternatively, a compound can be tested by applying it directly to a fungus or fungal cell, or expressing it therein, and monitoring the fungus or fungal cell for changes or decreases in growth, development, viability, pathogenicity, or alterations in gene expression. Thus, in one embodiment, the invention provides a method for determining whether a compound identified as an antibiotic candidate by an above method has antifungal activity, further comprising: contacting a fungus or fungal cells with the antifungal candidate and detecting a decrease in the growth, viability, or pathogenicity of the fungus or fungal cells.

By decrease in growth, is meant that the antifungal candidate causes at least a 10% decrease in the growth of the fungus or fungal cells, as compared to the growth of the fungus or fungal cells in the absence of the antifungal candidate. By a decrease in viability is meant that at least 20% of the fungal cells, or portion of the fungus contacted with the antifungal candidate are nonviable. Preferably, the growth or viability will be decreased by at least 40%. More preferably, the growth or viability will be decreased by at least 50%, 75% or at least 90% or more. Methods for measuring fungal growth and cell viability are known to those skilled in the art. By decrease in pathogenicity, is meant that the antifungal candidate causes at least a 10% decrease in the disease caused by contact of the fungal pathogen with its host, as compared to the disease caused in the absence of the antifungal candidate. Preferably, the disease will be decreased by at least 40%. More preferably, the disease will be decreased by at least 50%, 75% or at least 90% or more. Methods for measuring fungal disease are well known to those skilled in the art, and include such metrics as lesion formation, lesion size, sporulation, respiratory failure, and/or death.

The ability of a compound to inhibit HIS4 activity can be detected using in vitro enzymatic assays in which the disappearance of a substrate or the appearance of a product is directly or indirectly detected. HIS4 proteins catalyze the inter-conversion of L-histidinol, 2 NAD⁺ and H₂O to L-histidine and 2 NADH. Methods for measuring the progression of a HIS4 enzymatic reaction and/or a change in the concentration of one or more reactants are known to those of ordinary skill in the art and include, for example, monitoring the reduction of NAD⁺ according to the method described in Fink et al., 53 Genetics 445-59 (1966).

Thus, the invention provides a method for identifying a test compound as a candidate for an antibiotic, comprising: contacting L-histidinol and NAD⁺ with a HIS4 enzyme in the presence and absence of a test compound under conditions amenable to the HIS4 enzyme activity; and comparing the concentration for one or more of the substrates and/or products in the presence and absence of the test compound, wherein a difference in the presence of the test compound, relative to the absence, for any of the above reactants indicates that the test compound is a candidate for an antibiotic.

Active fragments of M. grisea HIS4 set forth in SEQ ID NO:2 are also useful in the methods of the invention. For example, an active polypeptide comprising at least 50 consecutive amino acid residues set forth in SEQ ID NO:2 and results in at least 10% of the activity of M. grisea HIS4 are useful in the methods of the invention. In addition, fragments of heterologous HIS4's are also useful in the methods of the invention. Active polypeptides having at least 10% of the activity of SEQ ID NO:2 and at least 50%, 60%, 70%, 80%, 90%, 95%, 96%, 97%, 98% or 99% sequence conservation with at least 50 consecutive amino acid residues of SEQ ID NO:2 are useful in the methods of the invention. Most preferably, the active polypeptide has at least 50% sequence conservation with at least 50 consecutive amino acid residues of SEQ ID NO:2 and at least 25%, 75% or at least 90% of the activity thereof.

Thus, the invention provides a method for identifying a test compound as a candidate for an antibiotic, comprising: contacting L-histidinol and NAD⁺ with a HIS4 enzyme in the presence and absence of a test compound under conditions amenable to the HIS4 enzyme activity, wherein the HIS4 enzyme is selected from the group consisting of: a polypeptide consisting essentially of SEQ ID NO:2, a polypeptide having at least 40% sequence conservation with the M. grisea HIS4 set forth in SEQ ID NO:2 and having at least 10% of the activity thereof, a polypeptide comprising at least 50 consecutive amino acids of M. grisea HIS4 set forth in SEQ ID NO:2 and having at least 0.10% of the activity thereof, and a polypeptide consisting of at least 50 amino acids and having at least 50% sequence conservation with M. grisea HIS4 set forth in SEQ ID NO:2 and having at least 10% of the activity thereof; and comparing the concentration for one or more of the substrates and/or products in the presence and absence of the test compound, wherein a difference in concentration in the presence of the test compound, relative to the absence, for any of the above reactants indicates that the test compound is a candidate for an antibiotic.

For in vitro enzymatic assays, a HIS4 protein and derivatives thereof are isolated from a fungus or may be recombinantly produced in and isolated from an archael, bacterial, fungal, or other eukaryotic cell culture. Preferably these proteins are produced using an E. coli, yeast, or filamentous fungal expression system using methods known to those skilled in the art.

The invention also provides cell-based assays. In one embodiment, the invention provides a method for identifying a test compound as a candidate for an antibiotic, comprising: a) measuring the expression or activity of a HIS4 in a cell, cells, tissue, or an organism in the absence of a test compound; b) contacting the cell, cells, tissue, or organism with the test compound and measuring the expression or activity of the HIS4 in the cell, cells, tissue, or organism; and c) comparing the expression or activity of the HIS4 in steps (a) and (b), wherein an altered expression or activity in the presence of the test compound indicates that the compound is a candidate for an antibiotic.

Expression of HIS4 can be measured by detecting the HIS4 primary transcript or mRNA, HIS4 polypeptide, or enzymatic activity of HIS4 polypeptide. Methods for detecting the expression of RNA and proteins are known to those skilled in the art. (Current Protocols in Molecular Biology, Ausubel et al., eds., Greene Publishing & Wiley-Interscience, New York, (1995)). The method of detection is not critical to the present invention. Methods for detecting HIS4 RNA include, but are not limited to, amplification assays such as quantitative reverse transcriptase-PCR, and/or hybridization assays such as Northern analysis, dot blots, slot blots, in-situ hybridization, transcriptional fusions using a HIS4 promoter fused to a reporter gene, DNA assays, and microarray assays.

Methods for detecting protein expression include, but are not limited to, immunodetection methods such as Western blots, ELISA assays, polyacrylamide gel electrophoresis, mass spectroscopy, and enzymatic assays. Also, any reporter gene system may be used to detect HIS4 protein expression. For detection using gene reporter systems, a polynucleotide encoding a reporter protein is fused in frame with HIS4 coding region so as to produce a chimeric polypeptide. Methods for using reporter systems are known to those skilled in the art.

Chemicals, compounds, or compositions identified by the above methods as modulators of HIS4 expression or activity can then be used to control fungal growth. Diseases such as rusts, mildews, and blights spread rapidly once established. Fungicides are thus routinely applied to growing and stored crops as a preventive measure, generally as foliar sprays or seed dressings. For example, compounds that inhibit fungal growth can be applied to a fungus or expressed in a fungus to prevent fungal growth. Thus, the invention provides a method for inhibiting fungal growth, comprising contacting a fungus with a compound identified by the methods of the invention as having antifungal activity.

Antifungals and antifungal inhibitor candidates identified by the methods of the invention can be used to control the growth of undesired fungi, including ascomycota, zygomycota, basidiomycota, chytridiomycota, and lichens. Examples of undesired fungi include, but are not limited to Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Monilinia fructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), diseases of animals such as infections of lungs, blood, brain, skin, scalp, nails or other tissues (Aspergillus fumigatus, Aspergillus sp., Fusraium sp., Trichophyton sp., Epidermophyton sp., and Microsporum sp., and the like).

Also provided in the invention are methods of screening for an antibiotic by determining the in vivo activity of a test compound against two separate fungal organisms, wherein the fungal organisms comprise a first form of a HIS4 and a second form of the HIS4, respectively. In the methods of the invention, at least one of the two forms of the HIS4 has at least 10% of the activity of the polypeptide set forth in SEQ ID NO:2. The methods comprise comparing the growth of the two organisms in the presence of the test compound relative to their respective controls without the test compound. A difference in growth between the two organisms in the presence of the test compound indicates that the test compound is a candidate for an antibiotic.

Forms of a HIS4 useful in the methods of the invention are selected from the group consisting of: a nucleic acid encoding SEQ ID NO:2; a nucleic acid encoding a polypeptide consisting essentially of SEQ ID NO:2; the nucleic acid set forth in SEQ ID NO:1; the nucleic acid set forth in SEQ ID NO:1 comprising a mutation either reducing or abolishing HIS4 protein activity; the nucleic acid set forth in SEQ ID NO:3; the nucleic acid set forth in SEQ ID NO:3 comprising a mutation either reducing or abolishing HIS4 protein activity; a nucleic acid encoding a heterologous HIS4; and a nucleic acid encoding a heterologous HIS4 comprising a mutation either reducing or abolishing HIS4 protein activity. Any combination of two different forms of the HIS4 genes listed above are useful in the methods of the invention, with the caveat that at least one of the forms of the HIS4 has at least 10% of the activity of the polypeptide set forth in SEQ ID NO:2.

Thus, in one embodiment, the invention provides a method for identifying a test compound as a candidate for an antibiotic, comprising: providing an organism having a first form of a HIS4; providing an organism having a second form of the HIS4; and determining the growth of the organism having the first form of the HIS4 and the growth of the organism having the second form of the HIS4 in the presence of the test compound, wherein a difference in growth between the two organisms in the presence of the test compound indicates that the test compound is a candidate for an antibiotic. It is recognized in the art that the optional determination of the growth of the organism having the first form of the HIS4 and the growth of the organism having the second form of the HIS4 in the absence of any test compounds is performed to control for any inherent differences in growth as a result of the different genes. Growth and/or proliferation of an organism are measured by methods well known in the art such as optical density measurements, and the like. In a preferred embodiment, the organism is Magnaporthe grisea.

In another embodiment, the invention provides a method for identifying a test compound as a candidate for an antibiotic, comprising: providing an organism having a first form of a HIS4; providing a comparison organism having a second form of the HIS4; and determining the pathogenicity of the organism having the first form of the HIS4 and the organism having the second form of the HIS4 in the presence of the test compound, wherein a difference in pathogenicity between the two organisms in the presence of the test compound indicates that the test compound is a candidate for an antibiotic. In an alternate embodiment of the invention, the pathogenicity of the organism having the first form of the HIS4 and the organism having the second form of the HIS4 in the absence of any test compounds is determined to control for any inherent differences in pathogenicity as a result of the different genes. Pathogenicity of an organism is measured by methods well known in the art such as lesion number, lesion size, sporulation, and the like. In a preferred embodiment the organism is Magnaporthe grisea.

In one embodiment of the invention, the first form of a HIS4 is SEQ ID NO:1 or SEQ ID NO:3, and the second form of the HIS4 is a HIS4 that confers a growth conditional phenotype (i.e. a histidine requiring phenotype) and/or a hypersensitivity or hyposensitivity phenotype on the organism. In a related embodiment of the invention, the second form of the HIS4 is SEQ ID NO:1 comprising a transposon insertion that reduces activity. In a related embodiment of the invention, the second form of a HIS4 is SEQ ID NO:1 comprising a transposon insertion that abolishes activity. In a related embodiment of the invention, the second form of the HIS4 is SEQ ID NO:3 comprising a transposon insertion that reduces activity. In a related embodiment of the invention, the second form of the HIS4 is SEQ ID NO:3 comprising a transposon insertion that abolishes activity. In a related embodiment of the invention, the second form of the HIS4 is Neurospora crassa histidinol dehydrogenase. In a related embodiment of the invention; the second form of the HIS4 is Candida albicans histidinol dehydrogenase. In a related embodiment of the invention, the second form of the HIS4 is Pichia pastoris histidinol dehydrogenase.

In another embodiment of the invention, the first form of an HIS4 is Neurospora crassa histidinol dehydrogenase and the second form of the HIS4 is Neurospora crassa histidinol dehydrogenase comprising a transposon insertion that reduces activity. In a related embodiment of the invention, the second form of the HIS4 is Neurospora crassa histidinol dehydrogenase comprising a transposon insertion that abolishes activity. In another embodiment of the invention, the first form of a HIS4 is Candida albicans histidinol dehydrogenase and the second form of the HIS4 is Candida albicans histidinol dehydrogenase comprising a transposon insertion that reduces activity. In a related embodiment of the invention, the second form of the HIS4 is Candida albicans histidinol dehydrogenase comprising a transposon insertion that abolishes activity. In yet another embodiment of the invention, the first form of a HIS4 is Pichia pastoris histidinol dehydrogenase and the second form of the HIS4 is Pichia pastoris histidinol dehydrogenase comprising a transposon insertion that reduces activity. In a related embodiment of the invention, the second form of the HIS4 is Pichia pastoris histidinol dehydrogenase comprising a transposon insertion that abolishes activity.

Conditional lethal mutants and/or antipathogenic mutants identify particular biochemical and/or genetic pathways given that at least one identified target gene is present in that pathway. Knowledge of these pathways allows for the screening of test compounds as candidates for antibiotics as inhibitors of the substrates, products, proteins and/or enzymes of the pathway. The invention provides methods of screening for an antibiotic by determining whether a test compound is active against the histidine biosynthetic pathway on which histidinol dehydrogenase functions. Pathways known in the art are found at the Kyoto Encyclopedia of Genes and Genomes and in standard biochemistry texts (See, e.g. Lehninger et al., Principles of Biochemistry, New York, Worth Publishers (1993)).

Thus, in one embodiment, the invention provides a method for screening for test compounds acting against the biochemical and/or genetic pathway or pathways in which histidinol dehydrogenase functions, comprising: providing an organism having a first form of a gene in the histidine biosynthetic pathway; providing an organism having a second form of the gene in the histidine biosynthetic pathway; and determining the growth of the two organisms in the presence of a test compound, wherein a difference in growth between the organism having the first form of the gene and the organism having the second form of the gene in the presence of the test compound indicates that the test compound is a candidate for an antibiotic. It is recognized in the art that the optional determination of the growth of the organism having the first form of the gene and the organism having the second form of the gene in the absence of any test compounds is performed to control for any inherent differences in growth as a result of the different genes. Growth and/or proliferation of an organism are measured by methods well known in the art such as optical density measurements, and the like. In a preferred embodiment, the organism is Magnaporthe grisea.

The forms of a gene in the histidine biosynthetic pathway useful in the methods of the invention include, for example, wild-type and mutated genes encoding histidinol phosphatase, imidazoleglycerol-phosphate dehydratase and histidinol-phosphate transaminase from any organism, preferably from a fungal organism, and most preferrably from M. grisea. The forms of a mutated gene in the histidine biosynthetic pathway comprise a mutation either reducing or abolishing protein activity. In one example, the form of a gene in the histidine biosynthetic pathway comprises a transposon insertion. Any combination of a first form of a gene in the histidine biosynthetic pathway listed above and a second form of the gene are useful in the methods of the invention, with the limitation that one of the forms of the gene in the histidine biosynthetic pathway has at least 10% of the activity of the corresponding M. grisea gene.

In another embodiment, the invention provides a method for screening for test compounds acting against the biochemical and/or genetic pathway or pathways in which histidinol dehydrogenase functions, comprising: providing an organism having a first form of a gene in the histidine biosynthetic pathway; providing an organism having a second form of the gene in the histidine biosynthetic pathway; and determining the pathogenicity of the two organisms in the presence of the test compound, wherein a difference in pathogenicity between the organism having the first form of the gene and the organism having the second form of the gene in the presence of the test compound indicates that the test compound is a candidate for an antibiotic. In an optional embodiment of the inventon, the pathogenicity of the two organisms in the absence of any test compounds is determined to control for any inherent differences in pathogenicity as a result of the different genes. Pathogenicity of an organism is measured by methods well known in the art such as lesion number, lesion size, sporulation, and the like. In a preferred embodiment the organism is Magnaporthe grisea.

Thus, in an alternate embodiment, the invention provides a method for screening for test compounds acting against the biochemical and/or genetic pathway or pathways in which histidinol dehydrogenase functions, comprising: providing paired growth media containing a test compound, wherein the paired growth media comprise a first medium and a second medium and the second medium contains a higher level of histidine than the first medium; inoculating the first and the second medium with an organism; and determining the growth of the organism, wherein a difference in growth of the organism between the first and the second medium indicates that the test compound is a candidate for an antibiotic. In one embodiment of the invention, the growth of the organism is determined in the first and the second medium in the absence of any test compounds to control for any inherent differences in growth as a result of the different media. Growth and/or proliferation of the organism are measured by methods well known in the art such as optical density measurements, and the like. In a preferred embodiment, the organism is Magnaporthe grisea.

One embodiment of the invention is directed to the use of multi-well plates for screening of antibiotic compounds. The use of multi-well plates is a format that readily accommodates multiple different assays to characterize various compounds, concentrations of compounds, and fungal organisms in varying combinations and formats. Certain testing parameters for the screening method can significantly affect the identification of growth inhibitors, and thus can be manipulated to optimize screening efficiency and/or reliability. Notable among these factors are variable sensitivities of different mutants, increasing hypersensitivity with increasingly less permissive conditions, an apparent increase in hypersensitivity with increasing compound concentration, and other factors known to those in the art.

### EXPERIMENTAL

### Example 1

### Construction of Plasmids with a Transposon Containing a Selectable Marker Construction of Sif Transposon

Sif was constructed using the GPS3 vector from the GPS-M mutagenesis system from New England Biolabs, Inc. (Beverly, Mass.) as a backbone. This system is based on the bacterial transposon Tn7. The following manipulations were done to GPS3 according to Sambrook et al., Molecular Cloning, a Laboratory Manual, Cold Spring Harbor Laboratory Press (1989). The kanamycin resistance gene (npt) contained between the Tn7 arms was removed by EcoRV digestion. The bacterial hygromycin B phosphotransferase (hph) gene (Gritz & Davies, 25 Gene 179 (1983) (PMID: 6319235)) under control of the Aspergillus nidulans trpC promoter and terminator (Mullaney et al., 199 Mol. Gen. Genet. 37 (1985) (PMID: 3158796)) was cloned by a HpaI/EcoRV blunt ligation into the Tn7 arms of the GPS3 vector yielding pSif1. Excision of the ampicillin resistance gene (bla) from pSif1 was achieved by cutting pSif1 with XmnI and BglI followed by a T4 DNA polymerase treatment to remove the 3′ overhangs left by the BglI digestion and religation of the plasmid to yield pSif. Top 10F′ electrocompetent E. coli cells (Invitrogen) were transformed with ligation mixture according to manufacturer's recommendations. Transformants containing the Sif transposon were selected on LB agar (Sambrook et al., supra) containing 50 μg/ml of hygromycin B (Sigma Chem. Co., St. Louis, Mo.).

### Example 2

### Construction of a Fungal Cosmid Library

Cosmid libraries were constructed in the pcosKA5 vector (Hamer et al., 98 Proc. Nat'l. Acad. Sci. USA 5110 (2001) (PMID: 11296265)) as described in Sambrook et al. Cosmid libraries were quality checked by pulsed-field gel electrophoresis, restriction digestion analysis, and PCR identification of single genes.

### Example 3

### Construction of Cosmids with Transposon Insertion into Fungal Genes Sif Transposition into a Cosmid

Transposition of Sif into the cosmid framework was carried out as described by the GPS-M mutagenesis system (New England Biolabs, Inc.). Briefly, 2 μl of the 10×GPS buffer, 70 ng of supercoiled pSIF, 8-12 μg of target cosmid DNA were mixed and taken to a final volume of 20 μl with water. 1 μl of transposase (TnsABC) was added to the reaction and incubated for 10 minutes at 37° C. to allow the assembly reaction to occur. After the assembly reaction, 1 μl of start solution was added to the tube, mixed well, and incubated for 1 hour at 37° C. followed by heat inactivation of the proteins at 75° C. for 10 minutes. Destruction of the remaining untransposed pSif was completed by PISceI digestion at 37° C. for 2 hr followed by a 10 min incubation at 75° C. to inactivate the proteins. Transformation of Top10F′ electrocompetent cells (Invitrogen) was done according to manufacturers recommendations. Sif-containing cosmid transformants were selected by growth on LB agar plates containing 50 μg/ml of hygromycin B (Sigma Chem. Co.) and 100 μg/ml of Ampicillin (Sigma Chem. Co.).

### Example 4

### High Throughput Preparation and Verification of Transposon Insertion Into the M. grisea HIS4 Gene

E. coli strains containing cosmids with transposon insertions were picked to 96 well growth blocks (Beckman Co.) containing 1.5 ml of TB (Terrific Broth, Sambrook et al., supra) supplemented with 50 μg/ml of ampicillin. Blocks were incubated with shaking at 37° C. overnight. E. coli cells were pelleted by centrifugation and cosmids were isolated by a modified alkaline lysis method (Marra et al., 7 Genome Res. 1072 (1997) (PMID: 9371743)). DNA quality was checked by electrophoresis on agarose gels. Cosmids were sequenced using primers from the ends of each transposon and commercial dideoxy sequencing kits (Big Dye Terminators, Perkin Elmer Co.). Sequencing reactions were analyzed on an ABI377 DNA sequencer (Perkin Elmer Co.).

The DNA sequences adjacent to the site of the transposon insertion were used to search DNA and protein databases using the BLAST algorithms (Altschul et al., supra). A construct having the SIF transposon insertion into the Magnaporthe grisea HIS4 gene was chosen for further analysis and designated cpgmra0037002c06.

### Example 5

### Preparation of HIS4 Cosmid DNA and Transformation of Magnaporthe grisea

Cosmid DNA from the HIS4 transposon tagged cosmid clone was prepared using QIAGEN Plasmid Maxi Kit (Qiagen), and digested by PI-PspI (New England Biolabs, Inc.). Fungal electro-transformation was performed essentially as described (Wu et al., 10 MPMI 700 (1997)). Briefly, M. grisea strain Guy 11 was grown in complete liquid media (Talbot et al., 5 Plant Cell 1575 (1993) (PMID: 8312740)) shaking at 120 rpm for 3 days at 25° C. in the dark. Mycelia was harvested and washed with sterile H₂O and digested with 4 mg/ml beta-glucanase (InterSpex) for 4-6 hr to generate protoplasts. Protoplasts were collected by centrifugation and resuspended in 20% sucrose at a concentration of 2×10⁸ protoplasts/ml. 50 μl of protoplast suspension was mixed with 10-20 μg of the cosmid DNA and pulsed using a Gene Pulser II instrument (BioRad) set with the following parameters: 200 ohm, 25 μF, and 0.6 kV. Transformed protoplasts were regenerated in complete agar media (Talbot et al., supra) with the addition of 20% sucrose for one day, then overlayed with CM agar media containing hygromycin B (250 μg/ml) to select transformants. Transformants were screened for homologous recombination events in the target gene by PCR (Hamer et al., supra). Two independent strains were identified and are hereby referred to as K1-38 and K1-48.

### Example 6

### Effect of Transposon Insertion into HIS4 on Magnaporthe Pathogenicity

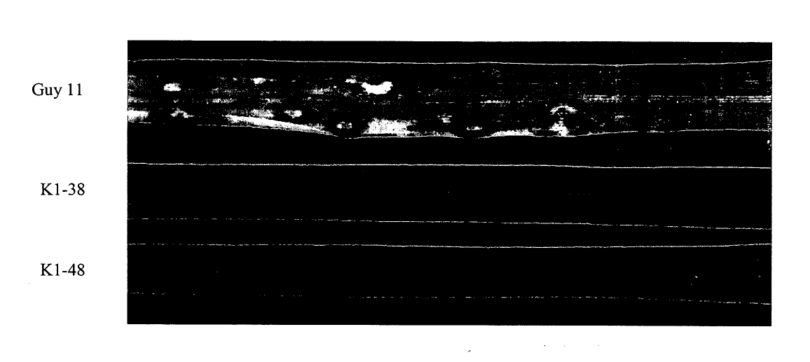

The target fungal strains, K1-38, and K1-48, obtained in Example 5 and the wild-type strain, Guy11, were subjected to a pathogenicity assay to observe infection over a 1-week period. Rice infection assays were performed using Indica rice cultivar CO₃₉ essentially as described in Valent et al. (Valent et al., 127 Genetics 87 (1991) (PMID: 2016048)). All three strains were grown for spore production on complete agar media. Spores were harvested and the concentration of spores adjusted for whole plant inoculations. Two-week-old seedlings of cultivar CO39 were sprayed with 12 ml of conidial suspension (5×10⁴ conidia per ml in 0.01% Tween-20 solution). The inoculated plants were incubated in a dew chamber at 27° C. in the dark for 36 hr, and transferred to a growth chamber (27° C., 12 hr/21° C., 12 hrs at 70% humidity) for an additional 5.5 days. Leaf samples were taken at 3, 5, and 7 days post-inoculation and examined for signs of successful infection (i.e. lesions). FIG. 1 shows the effects of HIS4 gene disruption on Magnaporthe infection at five days post-inoculation.

### Example 7

### Cloning Expression, and Isolation of Recombinant HIS4

The following is a protocol to obtain an isolated HIS4 protein or protein fragment.

Cloning and Expression Strategies:

A HIS4 encoding nucleic acid is cloned into E. coli (pET vectors-Novagen), Baculovirus (Pharmingen) and Yeast (Invitrogen) expression vectors containing His/fusion protein tags, and the expression of recombinant protein is evaluated by SDS-PAGE and Western blot analysis.

Extraction:

Extract recombinant protein from 250 ml cell pellet in 3 ml of an extraction buffer by sonicating 6 times, with 6 sec pulses at 4° C. Centrifuge extract at 15000×g for 10 min and collect supernatant.

Isolation:

Isolate recombinant protein by Ni-NTA affinity chromatography (Qiagen). Purification protocol (perform all steps at 4° C.):
Use 3 ml Ni-beads
Equilibrate column with the buffer
Load protein extract
Wash with the equilibration buffer
Elute bound protein with 0.5M imidazole

Assess biological activity of the recombinant HIS4 protein by activity assay such as by monitoring the reduction of NAD⁺ according to the method described by Finke et al., 53 Genetics 445-59 (1966), herein incorporated by reference in its entirety. The assay procedure is as follows:
Incubate the recombinant HIS4 enzyme with 50 μmol Tris HCl (pH 9.0), 0.4 μmol NAD⁺, and 2 μmol L-histidinol in a total volume of 35011.
Monitor the reduction of NAD⁺ in the incubation reaction at 340 nm.

### Example 8

### Assays for Screening Test Compounds for Binding/Inhibition of Isolated HIS4 Polypeptide

The following are protocols to identify test compounds that bind/inhibit isolated HIS4 protein.

Assay 1:

Test compounds are immobilized on a supportive medium.
Radioactively labeled HIS4 polypeptide is prepared by expressing the HIS4 polypeptide as described in Example 7 in the presence of radioactively labeled methionine (³⁵S-methionine, Amersham).
Screening for inhibitors is performed by incubating the radioactively labeled HIS4 polypeptide with the immobilized test compounds.
The wells are washed to remove excess labeled polypeptide and scintillation fluid (SCINTIVERSE, Fisher Scientific) is added to each well.
The plates are read in a microplate scintillation counter.

Candidate compounds are identified as wells with higher radioactivity as compared to control wells with no test compound added.

Assay 2:

Incubate the recombinant HIS4 enzyme in the presence and absence of test compounds in 50 μmol Tris HCl (pH 9.0), 0.41 mol NAD⁺, and 2 μmol L-histidinol in a total volume of 350 μl.
Monitor the reduction of NAD⁺ in the incubation reactions at 340 nm.
   A decreased rate of loss of NAD⁺ in the presence relative to the absence of a test compound indicates that the compound is a candidate antibiotic.

Additionally, an isolated polypeptide comprising 10-50 amino acids from the M. grisea HIS4 is screened in the same way. A polypeptide comprising 10-50 amino acids is generated by subcloning a portion of the HIS4 encoding nucleic acid into a protein expression vector that adds a His-Tag when expressed (see Example 7). Oligonucleotide primers are designed to amplify a portion of the HIS4 coding region using the polymerase chain reaction amplification method. The DNA fragment encoding a polypeptide of 10-50 amino acids is cloned into an expression vector, expressed in a host organism and isolated as described in Example 7 above.

Test compounds that bind and/or inhibit the HIS4 polypeptide are further tested for antibiotic activity. M. grisea is grown as described for spore production on oatmeal agar media (Talbot et al., supra). Spores are harvested into minimal media to a concentration of 2×10⁵ spores/ml and the culture is divided. Id. The test compound is added to one culture to a final concentration of 20-100 μg/ml. Solvent only is added to the second culture. The plates are incubated at 25° C. for 7 days and optical density measurements at 590 nm are taken daily. The growth curves of the solvent control sample and the test compound sample are compared. A test compound is an antibiotic candidate if the growth of the culture containing the test compound is less than the growth of the control culture.

Test compounds that bind and/or inhibit the HIS4 polypeptide are further tested for antipathogenic activity. M. grisea is grown as described for spore production on oatmeal agar media (Talbot et al., supra). Spores are harvested into water with 0.01% Tween 20 to a concentration of 5×10⁴ spores/ml and the culture is divided. Id. The test compound is added to one culture to a final concentration of 20-100 μg/ml. Solvent only is added to the second culture. Rice infection assays are performed using Indica rice cultivar CO₃₉ essentially as described in Valent et al., supra. 24-two-week-old seedlings of cultivar CO₃₉ are sprayed with 12 ml of conidial suspension. The inoculated plants are incubated in a dew chamber at 27° C. in the dark for 36 hr, and transferred to a growth chamber (27° C., 12 hr/21° C., 12 hr at 70% humidity) for an additional 5.5 days. Leaf samples are examined at 5 days post-inoculation to determine the extent of pathogenicity as compared to the control samples.

Alternatively, antipathogenic activity can be assessed using an excised leaf pathogenicity assay. Spore suspensions are prepared in water only to a concentration of 5×10⁴ spores/ml and the culture is divided. The test compound is added to one culture to a final concentration of 20-100 μg/ml. Solvent only is added to the second culture. Detached leaf assays are performed by excising 1 cm segments of rice leaves from Indica rice cultivar CO₃₉ and placing them on 1% agarose in water. 10 μl of each spore suspension is place on the leaf segments and the samples are incubated at 25° C. for 5 days in the dark. Leaf samples are examined at 5 days post-inoculation to determine the extent of pathogenicity as compared to the control samples.

The effect of each of the test compounds on pathogenicity for the mutant and wild-type strains relative to the solvent controls is compared. Compounds that show differential degrees of pathogenicity between the mutant and the wild-type strains relative to the solvent controls (e.g. differences in lesion number, lesion size, or the competency of a lesion to sporulate) are identified as potential fungicidal compounds. For example, a reduction in the pathogenicity of the wild-type strain but not the mutant strain in the presence relative to the absence of the test compound suggests that the target of the test compound is the HIS4 gene product.

### Example 9

### Assays for Testing Compounds for Alteration of HIS4 Gene Expression

Magnaporthe grisea fungal cells are grown under standard fungal growth conditions that are well known and described in the art. Wild-type M. grisea spores are harvested from cultures grown on complete agar or oatmeal agar media after growth for 10-13 days in the light at 25° C. using a moistened cotton swab. The concentration of spores is determined using a hemacytometer and spore suspensions are prepared in a minimal growth medium to a concentration of 2×10⁵ spores/ml. 25 ml cultures are prepared to which test compounds will be added at various concentrations. A culture with no test compound present is included as a control. The cultures are incubated at 25° C. for 3 days after which test compound or solvent only control is added. The cultures are incubated an additional 6 hr. Fungal mycelia is harvested by filtration through Miracloth (CalBiochem, La Jolla, Calif.), washed with water, and frozen in liquid nitrogen. Total RNA is extracted with TRIZOL Reagent using the methods provided by the manufacturer (Life Technologies, Rockville, Md.). Expression is analyzed by Northern analysis of the RNA samples as described (Sambrook et al.; supra) using a radiolabeled fragment of the HIS4 encoding nucleic acid as a probe. Test compounds resulting in an altered level of HIS4 mRNA relative to the untreated control sample are identified as candidate antibiotic compounds.

Test compounds identified as inhibitors of HIS4 gene expression are further tested for antibiotic activity by measuring the effect of the test compound on Magnaporthe grisea growth and/or pathogenicity as described above in Example 8.

### Example 10

### In Vivo Cell Based Assay Screening Protocol with a Fungal Strain Containing a Mutant Form of HIS4 with Reduced or No Activity

The effect of test compounds on the growth of wild-type fungal cells and mutant fungal cells having a mutant HIS4 gene is measured and compared as follows. Magnaporthe grisea fungal cells containing a mutant form of the HIS4 gene that has reduced activity or lacks activity, for example a HIS4 gene containing a transposon insertion, are grown under standard fungal growth conditions that are well known and described in the art. Magnaporthe grisea spores are harvested from cultures grown on complete agar medium containing L-histidine (Sigma) after growth for 10-13 days in the light at 25° C. using a moistened cotton swab. The concentration of spores is determined using a hemacytometer and spore suspensions are prepared in a minimal growth medium containing L-hisitidine to a concentration of 2×10⁵ spores per ml. Approximately 4×10⁴ spores are added to each well of 96-well plates to which a test compound is added (at varying concentrations). The total volume in each well is 200 μl. Wells with no test compound present (growth control), and wells without cells are included as controls (negative control). The plates are incubated at 25° C. for seven days and optical density measurements at 590 nm are taken daily. Wild-type cells are screened under the same conditions.

The effect of each of the test compounds on the mutant and wild-type fungal cells is measured against the growth control and the percent of inhibition is calculated as the OD₅₉₀ (fungal strain plus test compound)/OD₅₉₀ (growth control)×100. The percent of growth inhibition in the presence of the test compound on the mutant and wild-type fungal strains are compared. Compounds that show differential growth inhibition between the mutant and the wild-type cells are identified as potential antifungal compounds. Similar protocols may be found in Kirsch & DiDomenico, 26 Biotechnology 177 (1994) (PMID: 7749303)).

Test compounds that produce a differential growth response between the mutant and wild-type fungal strains are further tested for antipathogenic activity as described in Example 8.

### Example 11

### In Vivo Cell Based Assay Screening Protocol with a Fungal Strain Containing a Mutant Form of a Histidine Biosynthetic Gene with Reduced or No Activity

The effect of test compounds on the growth of wild-type fungal cells and mutant fungal cells having a mutant form of a gene in the histidine biosynthetic pathway is measured and compared as follows. Magnaporthe grisea fungal cells containing a mutant form of a gene with reduced or no activity in the histidine biosynthetic pathway (e.g. histidinol phosphatase, imidazoleglycerol-phosphate dehydratase or histidinol-phosphate transaminase having a transposon insertion) are grown under standard fungal growth conditions that are well known and described in the art. Magnaporthe grisea spores are harvested from cultures grown on complete agar medium containing L-histidine (Sigma) after growth for 10-13 days in the light at 25° C. using a moistened cotton swab. The concentration of spores is determined using a hemacytometer and spore suspensions are prepared in a minimal growth medium containing L-histidine to a concentration of 2×10⁵ spores per ml.

Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. Wild-type cells are screened under the same conditions.

The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of each of the test compounds on the mutant and the wild-type cells are compared. Compounds that show differential growth inhibition between the mutant and the wild-type cells are identified as potential antifungal compounds. Similar protocols may be found in Kirsch & DiDomenico, supra.

Test compounds that produce a differential growth response between the mutant and wild-type fungal strains are further tested for antipathogenic activity as described in Example 8.

### Example 12

### In Vivo Cell Based Assay Screening Protocol with a Fungal Strain Containing a Heterologous Histidinol Dehydrogenase Gene

The effect of test compounds on the growth of wild-type fungal cells and fungal cells lacking a functional endogenous histidinol dehydrogenase gene and containing a heterologous histidinol dehydrogenase gene is measured and compared as follows. Wild-type M. grisea fungal cells and M. grisea fungal cells lacking an endogenous histidinol dehydrogenase gene and containing a heterologous histidinol dehydrogenase gene from Candida albicans (Genbank Accession No. 074712), having 47% sequence identity, are grown under standard fungal growth conditions that are well known and described in the art.

A M. grisea strain carrying a heterologous histidinol dehydrogenase gene is made as follows. A M. grisea strain is made with a nonfunctional endogenous histidinol dehydrogenase gene, such as one containing a transposon insertion in the native gene that abolishes protein activity. A construct containing a heterologous histidinol dehydrogenase gene is made by cloning a heterologous histidinol dehydrogenase gene, such as from Candida albicans, into a fungal expression vector containing a trpC promoter and terminator (e.g. Carroll et al., 41 Fungal Gen. News Lett. 22 (1994) (describing pCB1003) using standard molecular biology techniques that are well known and described in the art (Sambrook et al., supra). The vector construct is used to transform the M. grisea strain lacking a functional endogenous histidinol dehydrogenase gene. Fungal transformants containing a functional histidinol dehydrogenase gene are selected on minimal agar medium lacking L-histidine, as only transformants carrying a functional histidinol dehydrogenase gene grow in the absence of L-histidine.

Wild-type strains of M. grisea and strains containing a heterologous form of histidinol dehydrogenase are grown under standard fungal growth conditions that are well known and described in the art. M. grisea spores are harvested from cultures grown on complete agar medium after growth for 10-13 days in the light at 25° C. using a moistened cotton swab. The concentration of spores is determined using a hemacytometer and spore suspensions are prepared in a minimal growth medium to a concentration of 2×10⁵ spores per ml.

Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for 7 days and optical density measurements at 590 nm are taken daily. The effect of each compound on the wild-type and heterologous fungal strains is measured against the growth control and the percent of inhibition is calculated as the OD₅₉₀ (fungal strain plus test compound)/OD₅₉₀ (growth control)×100. The percent of growth inhibition as a result of each of the test compounds on the wild-type and heterologous fungal strains are compared. Compounds that show differential growth inhibition between the wild-type and heterologous strains are identified as potential antifungal compounds with specificity to the native or heterologous histidinol dehydrogenase gene products. Similar protocols may be found in Kirsch & DiDomenico, supra.

Test compounds that produce a differential growth response between the strain containing a heterologous gene and strain containing a fungal gene are further tested for antipathogenic activity as described in Example 8.

### Example 13

### Verification of HIS4 Gene Function by Analysis of Nutritional Requirements

The fungal strains, K1-38 and K1-48, containing the HIS4 disrupted gene obtained in Example 5 and the wildtype strain were transferred to minimal agar media. Neither strain containing the disrupted HIS4 gene grew on the minimal agar media. The fungal strains are analyzed for their nutritional requirement for histidine by growing each strain in aminoculating fluid consisting of 0.05% Phytagel, 0.03% Pluronic F68, 1% glucose, 23.5 mM NaNO₃, 6.7 mM KCl, 3.5 mM Na₂SO₄, 11.0 mM KH₂PO₄, 0.01% p-iodonitrotetrazolium violet, 0.1 mM MgCl₂, 1.0 mM CaCl₂ and trace elements, pH adjusted to 6.0 with NaOH. Final concentrations of trace elements are: 7.6 μM ZnCl₂, 2.5 μM MnCl₂.4H₂₀, 1.8 μM FeCl₂.4H₂O, 0.71 μM CoCl₂.6H₂O, 0.64 μM CuCl₂.2H₂O, 0.62 μM Na₂MoO₄, 18 μM H₃BO₃. Spores for each strain are harvested into the inoculating fluid with and without the addition of 4 mM L-histidine (Sigma). The spore concentrations are adjusted to 2×10⁵ spores/ml. 200 μl of spore suspension are deposited into each well of the microtiter plates. The plates are incubated at 25° C. for 7 days. Optical density (OD) measurements at 590 nm are taken daily. Growth in the presence but not the absence of L-histidine confirms that disruption of the HIS4 gene blocks histidine biosynthesis in the mutant strains.

### Example 14

### Pathway Specific In Vivo Assay Screening Protocol

Compounds are tested as candidate antibiotics as follows. Magnaporthe grisea fungal cells are grown under standard fungal growth conditions that are well known and described in the art. Wild-type M. grisea spores are harvested from cultures grown on oatmeal agar media after growth for 10-13 days in the light at 25° C. using a moistened cotton swab. The concentration of spores is determined using a hemocytometer and spore suspensions are prepared in a minimal growth medium and a minimal growth medium containing L-histidine (Sigma) to a concentration of 2×10⁵ spores per ml. The minimal growth media contains carbon, nitrogen, phosphate, and sulfate sources, and magnesium, calcium, and trace elements (for example, see inoculating fluid in Example 13). Spore suspensions are added to each well of a 96-well microtiter plate (approximately 4×10⁴ spores/well). For each well containing a spore suspension in minimal media, an additional well is present containing a spore suspension in minimal medium containing L-histidine.

Test compounds are added to wells containing spores in minimal media and minimal media containing L-histidine. The total volume in each well is 200 μl. Both minimal media and L-histidine containing media wells with no test compound are provided as controls. The plates are incubated at 25° C. for 7 days and optical density measurements at 590 nm are taken daily. A compound is identified as a candidate for an antibiotic acting against the L-histidine biosynthetic pathway when the observed growth in the well containing minimal media is less than the observed growth in the well containing L-histidine as a result of the addition of the test compound. Similar protocols may be found in Kirsch & DiDomenico, supra.

Test compounds that are identified as candidates for an antibiotic are further tested for antipathogenic activity as described in Example 8.

Published references and patent publications cited herein are incorporated by reference as if terms incorporating the same were provided upon each occurrence of the individual reference or patent document. While the foregoing describes certain embodiments of the invention, it will be understood by those skilled in the art that variations and modifications may be made that will fall within the scope of the invention. The foregoing examples are intended to exemplify various specific embodiments of the invention and do not limit its scope in any manner.

1. A method for identifying a test compound as a candidate for an antibiotic, comprising:
a) contacting a polypeptide with a test compound, wherein said polypeptide is selected from the group consisting of:
i) a non-fungal histidinol dehydrogenase polypeptide;
ii) a fungal histidinol dehydrogenase polypeptide,
iii) a Magnaporthe histidinol dehydrogenase polypeptide;
iv) a polypeptide comprising SEQ ID NO:2;
v) a polypeptide consisting essentially of SEQ ID NO:2;
vi) a polypeptide having at least ten consecutive amino acids of SEQ ID NO:2;
vii) a polypeptide having at least 50% sequence identity with SEQ ID NO:2 and at least 10% of the activity of SEQ ID NO:2; and
viii) a polypeptide consisting of at least 50 amino acids having at least 50% sequence identity with SEQ ID NO:2 and at least 10% of the activity of SEQ ID NO:2; and
b) carrying out at least one assay selected from the group consisting of:
i) detecting the presence or absence of binding between the test compound and the histidinol dehydrogenase polypeptide, wherein binding indicates that the test compound is a candidate for an antibiotic; and
ii) monitoring the reduction of NAD+ in the presence and absence of the test compound, wherein a decreased rate of loss of NAD+ in the presence relative to the absence of the test compound indicates that the compound is a candidate for an antibiotic.

2. A method for identifying a test compound as a candidate for an antibiotic, comprising:
a) measuring the expression of a histidinol dehydrogenase in an organism, or a cell or tissue thereof, in the presence and absence of a test compound; and
b) comparing the expression of the histidinol dehydrogenase in the presence and absence of the test compound, wherein an altered expression in the presence of the test compound indicates that the test compound is a candidate for an antibiotic.

3. The method of claim 2, wherein the organism is a fungus.

4. The method of claim 2, wherein the organism is Magnaporthe.

5. The method of claim 2, wherein the histidinol dehydrogenase comprises SEQ ID NO:2.

6. The method of claim 2, wherein the expression of the histidinol dehydrogenase is measured by at least one of the following methods: detecting the histidinol dehydrogenase mRNA, detecting the histidinol dehydrogenase polypeptide, and detecting the histidinol dehydrogenase polypeptide enzyme activity.

7. A method for identifying a test compound as a candidate for an antibiotic comprising:
a) providing a fungal organism having a first form of a histidinol dehydrogenase;
b) providing a fungal organism having a second form of the histidinol dehydrogenase, wherein one of the first or the second form of the histidinol dehydrogenase has at least 10% of the activity of SEQ ID NO:2; and
c) carrying out at least one assay selected from the group consisting of:
i) determining the growth of the organism having the first form of the histidinol dehydrogenase and the organism having the second form of the histidinol dehydrogenase in the presence of a test compound, wherein a difference in growth between the two organisms in the presence of the test compound indicates that the test compound is a candidate for an antibiotic; and
ii) determining the pathogenicity of the organism having the first form of the adenylosuccinate synthase and the organism having the second form of a adenylosuccinate synthase in the presence of a test compound, wherein a difference in pathogenicity between the two organisms in the presence of the test compound indicates that the test compound is a candidate for an antibiotic.

8. The method of claim 7, wherein the fungal organism having the first form of the histidinol dehydrogenase and the fungal organism having the second form of the histidinol dehydrogenase are Magnaporthe;
wherein the first form of the histidinol dehydrogenase is selected from the group consisting of: fungal histidinol dehydrogenases and a polypeptide comprising SEQ ID NO:2; and
wherein the second form of the histidinol dehydrogenase is selected from the group consisting of: fungal histidinol dehydrogenases, a polypeptide comprising SEQ ID NO:2, a heterologous histidinol dehydrogenase, a nucleic acid sequence comprising SEQ ID NO:1 further comprising a transposon insertion that reduces or abolishes histidinol dehydrogenase activity, and SEQ ID NO:2 comprising a transposon insertion that reduces or abolishes adenylosuccinate synthase activity.


SEQUENCE LISTING (3 sequences)

SEQ ID NO: 1 - DNA, 2598 bp, Magnaportha grisea
atggagtcaacactaccgttgcctttcattgttgatgcctctgtcaacctcaatggcgaagctggcctctccaaggagcagcttgcttgtcttggcacgatattcttcgaggtcacgccgcagaatcttggtgacgtgaggagctttttacagccgggcacttctgccttcgaaccatatctggatgtgactcaactcgagtctgccaacgacatattgtccctacttgatagtggtgccaggaaggtctttgttaaaccggagcagttgaaggactatgaggagcacggctcaagggttggacaggctgttgacggaacctcattgcaggtctccgcagcagagaatggtctgctcgtgagcggcatagatgcgagcggtgatgtttcaacactggtccagcagttcaactcgaaaaagggctcacccctgttcatcaggccagcagacggtgccgatttggagctttgcgctgccttggcccgacaagtgcatgccactgttatcttgccgtcatcaaggctgactgcttgcaccaaagatgccaccggcgggaaggtttcgatatctaagctcttggcatcaaactggacttctgacagaggagacaagctgcttcctacggtggtcactgacgataatggaatcgccctgggactggtatacagcagcgaggagagcatcggagaggccctgcggacatgcacgggtgtctaccaaagtcgcaagcgtggtttatggtacaagggagccacttcaggagacactcaggagctggttcgaatctctcttgactgcgacaacgatgctctcaaattcgtcgttaggcaaaagggacgtttctgccacctcgaccagtttagctgctttggaaacctcggcggcattgccaagttggagcaaacactcacacaacgcagagagtcggctcctgcaggctcgtacactgccaggttgttttcagatgagaagcttttgagggccaagatcatggaggaggctgaggagctttgcgatgcaaagaccaaggagaacgttgcctttgaggccgctgaccttatctactttgcgctgacgaaggcggttgcttcgggcgtcagtttgagcgacatcgagaggaaccttgacgctaagagctggaaggtcaagcgtaggacgggagacgccaagggcaaatgggccgagaaggaaggtatcaagcctagtgcacccagtgcgctcgcaccagctcctgcgcctgctgctacagaggcgacgtctgacaggatcgcaatgaaggtcctcgacgtcagccagagctctgttgccgacatccaagaagccctgaagcgcccctcccaaaagtcgtccgacgccatcatgaagatcatcggccccatagtcgacgacgtccacacgaatggcgacaaggctgtcctgtcatacactcacaagttcgaaaaggcaacgtccctgacgtcacccgtcctcaaggcccccttccccgaggagatgatgcgcctgtcccccgagactgccaaggccatcgacatctcgttcgagaacatccgcaagttccacgcggcccagaaggaggacaagcccctgcgcgttgagaccatgcccggcgtcgtgtgcagccgcttcagtcggcccatcgagcgcgtgggcctgtacgtgcccggcggcacggccgtgctgccctctaccgccctcatgcttggcgtccccgccatggtggccggctgccagcgcatcgtgctcgcctccccgccgcgccaggacggcaccgtgactcccgagattgtctacgtcgcccacaaggtcggcgccgagagcatcgtgctcgccggtggcgcccaggcagtcgctgccatggcgtacggaaccgagagcgttaccaaggtcgacaagatcctgggcccaggtaaccagttcgtcacggcggctaagatgctagtcagcaacgacaccaacgccggcgtcggcatcgacatgcccgccggcccctctgaggtactcgtcattgccgactgcgacgccaacccggcctttgtcgcgtcggacctcctctcccaggccgagcacggcgtcgacagccaggtcgtcctcatcgccgtcgacctggacgaggcgggcctcaaggccatcgaggacgaggtccacaggcaggccatggcgctgcccagggtcgatattgtcaggggtagcatcaagcactcaatcaccatctcggtccgcaacatcgaggaggccatgcgcatcagcaacgactacgcaccggagcatctcatcctgcagctcaagaacgcagaggccgtcgtcgacatggtcatgaatgccggcagtgtgtttatcgggcagtggacgcctgagagcgttggcgattattctgccggtgtcaaccactcgctccctacatacggctacgcaaagcaatactcgggcgtcaaccttggctcgtttgtcaagcacataaccagctccaatctgactgcggacggtcttcgcaacgttggtgaggccgtcatgcaactggccaaggtcgaggagcttgaagcccacaggagggctgttagcattcgtatggaatacatgaacaagcaagccaaccagtag

SEQ ID NO: 2 - protein, 865 aa, Magnaportha grisea
MESTLPLPFIVDASVNLNGEAGLSKEQLACLGTIFFEVTPQNLGDVRSFLQPGTSAFEPYLDVTQLESANDILSLLDSGARKVFVKPEQLKDYEEHGSRVGQAVDGTSLQVSAAENGLLVSGIDASGDVSTLVQQFNSKKGSPLFIRPADGADLELCAALARQVHATVILPSSRLTACTKDATGGKVSISKLLASNWTSDRGDKLLPTVVTDDNGIALGLVYSSEESIGEALRTCTGVYQSRKRGLWYKGATSGDTQELVRISLDCDNDALKFVVRQKGRFCHLDQFSCFGNLGGIAKLEQTLTQRRESAPAGSYTARLFSDEKLLRAKIMEEAEELCDAKTKENVAFEAADLIYFALTKAVASGVSLSDIERNLDAKSWKVKRRTGDAKGKWAEKEGIKPSAPSALAPAPAPAATEATSDRIAMKVLDVSQSSVADIQEALKRPSQKSSDAIMKIIGPIVDDVHTNGDKAVLSYTHKFEKATSLTSPVLKAPFPEEMMRLSPETAKAIDISFENIRKFHAAQKEDKPLRVETMPGVVCSRFSRPIERVGLYVPGGTAVLPSTALMLGVPAMVAGCQRIVLASPPRQDGTVTPEIVYVAHKVGAESIVLAGGAQAVAAMAYGTESVTKVDKILGPGNQFVTAAKMLVSNDTNAGVGIDMPAGPSEVLVIADCDANPAFVASDLLSQAEHGVDSQVVLIAVDLDEAGLKAIEDEVHRQAMALPRVDIVRGSIKHSITISVRNIEEAMRISNDYAPEHLILQLKNAEAVVDMVMNAGSVFIGQWTPESVGDYSAGVNHSLPTYGYAKQYSGVNLGSFVKHITSSNLTADGLRNVGEAVMQLAKVEELEAHRRAVSIRMEYMNKQANQ

SEQ ID NO: 3 - DNA, 2688 bp, Magnaportha grisea
atggagtcaacactaccgttgcctttcattgttgatgcctctgtcaacctcaatggcgaagctggcctctccaaggagcagcttgcttgtcttggcacgatattcttcgaggtcacgccgcagaatcttggtgacgtgaggagctttttacagccgggcacttctgccttcgaaccatatctggatgtgactcaactcgagtctgccaacgacatattgtccctacttgatagtggtgccaggaaggtctttgttaaaccggagcagttgaaggactatgaggagcacggctcaagggttggacaggctgttgacggaacctcattgcaggtctccgcagcagagaatggtctgctcgtgagcggcatagatgcgagcggtgatgtttcaacactggtccagcagttcaactcgaaaaagggctcacccctgttcatcaggccagcagacggtgccgatttggagctttgcgctgccttggcccgacaagtgcatgccactgttatcttgccgtcatcaaggctgactgcttgcaccaaagatgccaccggcgggaaggtttcgatatctaagctcttggcatcaaactggacttctgacagaggagacaagctgcttcctacggtggtcactgacgataatggaatcgccctgggactggtatacagcagcgaggagagcatcggagaggccctgcggacatgcacgggtgtctaccaaagtcgcaagcgtggtttatggtacaagggagccacttcaggagacactcaggagctggttcgaatctctcttgactgcgacaacgatgctctcaaattcgtcgttaggcaaaagggacgtttctgccacctcgaccagtttagctgctttggaaacctcggcggcattgccaagttggagcaaacactcacacaacgcagagagtcggctcctgcaggctcgtacactgccaggttgttttcagatgagaagcttttgagggccaagatcatggaggaggctgaggagctttgcgatgcaaagaccaaggagaacgttgcctttgaggccgctgaccttatctactttgcgctgacgaaggcggttgcttcgggcgtcagtttgagcgacatcgagaggaaccttgacgctaagagctggaaggtcaagcgtaggacgggagacgccaagggcaaatgggccgagaaggaaggtatcaagcctagtgcacccagtgcgctcgcaccagctcctgcgcctgctgctacagaggcgacgtctgacaggatcgcaatgaaggtcctcgacgtcagccagagctctgttgccgacatccaagaagccctgaagcgcccctcccaaaagtcgtccgacgccatcatgaagatcatcggccccatagtcgacgacgtccacacgaatggcgacaaggctgtcctgtcatacactcacaagttcgaaaaggcaacgtccctgacgtcacccgtcctcaaggcccccttccccgaggagatgatgcgcctgtcccccgagactgccaaggccatcgacatctcgttcgagaacatccgcaagttccacgcggcccagaaggaggacaagcccctgcgcgttgagaccatgcccggcgtcgtgtgcagccgcttcagtcggcccatcgagcgcgtgggcctgtacgtgcccggcggcacggccgtgctgccctctaccgccctcatgcttggcgtccccgccatggtggccggctgccagcgcatcgtgctcgcctccccgccgcgccaggacggcaccgtgactcccgagattgtctacgtcgcccacaaggtcggcgccgagagcatcgtgctcgccggtggcgcccaggcagtcgctgccatggcgtacggaaccgagagcgttaccaaggtcgacaagatcctgggcccaggtaaccagttcgtcacggcggctaagatgctagtcagcaacgacaccaacgccggcgtcggcatcgacatgcccgccggcccctctgaggtactcgtcattgccgactgcgacgccaacccggcctttgtcgcgtcggacctcctctcccaggccgagcacggcgtcgacagccaggtcgtcctcatcgccgtcgacctggacgaggcgggcctcaaggccatcgaggacgaggtccacaggcaggccatggcgctgcccagggtcgatattgtcaggggtagcatcaagcactcaatcaccatctcggtccgcaacatcgaggaggccatgcgcatcagcaacgactacgcaccggagcatctcatcctgcagctcaagaacgcagaggccgtcgtcgacatggtcatgaatgccggcagtgtgtttatcgggcagtggacgcctgagagcgttggcgattattctgccggtgtcaaccactcgctccgtaagtctacccttgaaatatgcccatcaggggcaaaagtgcgagggaaattctcttgtctcaatgctaaccttttccccccttgaacagctacatacggctacgcaaagcaatactcgggcgtcaaccttggctcgtttgtcaagcacataaccagctccaatctgactgcggacggtcttcgcaacgttggtgaggccgtcatgcaactggccaaggtcgaggagcttgaagcccacaggagggctgttagcattcgtatggaatacatgaacaagcaagccaaccagtag